(19) United States
(12) Patent Application Publication
(10) Pub. No.: US 2005/0092641 A1
(43) Pub. Date: May 5, 2005

(54) Medicine container and packaging therefor

(76) Inventors: Andrew W. Marsden, Hingham, MA (US); Joseph C. Cacciola, Wrentham, MA (US)

Correspondence Address: BARNES & THORNBURG, 11 SOUTH MERIDIAN, INDIANAPOLIS, IN, 46204, US

(21) Appl. No.: 10/698,649

(22) Filed: Oct. 31, 2003

(51) Int. Cl. A61B 17/06

(52) U.S. Cl. 206/438; 206/503

(57) ABSTRACT

A monolithic container, for storing and dispensing medicines. The cup has a plurality of spaced-apart stepped increments and indicia for indicating capacities and content levels of the cup.

### BACKGROUND

The present disclosure relates to containers and container packaging, and in particular, to containers for storing and dispensing medicines. More particularly, the present disclosure relates to lidded containers.

Containers for storing and/or dispensing solid and/or liquid medicines are known. Generally, those containers are in the form of a variety of measuring devices, such as spoons or cups or similar devices.

### SUMMARY

According to the present disclosure, a container for storing and dispensing medicines includes a lid and a cup connected to the lid by a tether strip providing a living hinge. The cup has a generally conically-tapered configuration. The cup includes spaced-apart stepped increments indicating capacities and content levels of the cup.

In preferred embodiments, the cup, lid, and living hinge are made of a plastics material to form a monolithic container. The cup and lid cooperate to form a securing mechanism. The securing mechanism releasably secures the lid and the cup when at least one of the lid and cup is rotated about the living hinge and closed on the other.

The containers of the present disclosure, in an opened condition, are nestable. A nested stack of containers is insertable and sealable in a package. When nested, the cups form a stack and the lids are aligned to form a second stack alongside the stack of cups and are retained in that aligned second stack by the somewhat stiff but pliable tether strip.

Other aspects of the present disclosure will become apparent from the following descriptions when considered in conjunction with the accompanying drawings.

### BRIEF DESCRIPTION OF THE DRAWINGS

The detailed description particularly refers to the following figures in which:

FIG. 1 is a perspective view of a container, according to the principles of the present disclosure;

FIG. 2 is a top plan view of the container of FIG. 1;

FIG. 3 is a sectional view of the container of FIGS. 1 and 2 after movement of the lid to a closed position closing an open mouth of a medicine storage region formed in the cup;

FIG. 4 is an elevation view of a collection of containers of the type shown in FIG. 1 showing a first stack of nested cups and showing alignment of the companion lids in a second stack owing to sufficient stiffness of the pliable lid tethers interconnecting each cup and lid pair; and

FIG. 5 is a top plan view of the collection of containers of FIG. 4 located in a cup storage region formed in a package.

### DETAILED DESCRIPTION

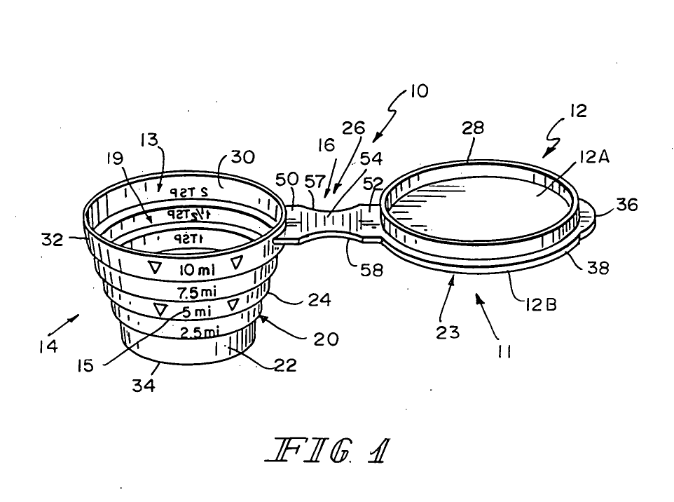

An embodiment of a container 10, for storing and dispensing medicines, is shown, for example, in FIG. 1. Container 10 includes a lid 12 and a cup 14 connected by a monolithically incorporated tether providing a living hinge 16. The cup 14 has a generally conically-tapered configuration 18. Cup 14 is formed to include a medicine storage chamber 19 to provide a receptacle for the liquid or other medicines and lid 12 is configured to close on and open cup 14. Cup 14 also has spaced-apart stepped increments 20 defining an outer surface 22 of side wall 24. Stepped increments 20 indicate medicine capacities of medicine storage chamber 19, for example, one teaspoon (1 tsp) or five millimeters (5 ml). Stepped increments 20 may also indicate or assist in indicating medicine content levels.

A securing mechanism 26 is monolithically incorporated with the container 10. Securing mechanism 26, as shown in FIG. 1, includes interlocking elements 28, 30 that are adapted to secure lid 12 and cup 14 when one or more of lid 12 and cup 14 is rotated about living hinge 16 and closed on the other.

In the illustrative embodiment 28 is a flange in lid 12 and interlocking element 30 is a rim defining an opening 13 into medicine storage chamber 19. Flange 28 may be on an underside 12A of lid 12. It is within the scope of this disclosure to locate flange 28 on cup 14 and recess 30 on lid 12. Although container 10 was designed for liquid which suggests that securing mechanism 26 seals lid 12 to cup 14, it may also be used for pills or other solids. In which case, securing mechanism 26 need not form a seal, but just secure any portion of lid 12 to cup 14 to retain lid 12 in a mounted position on cup 14.

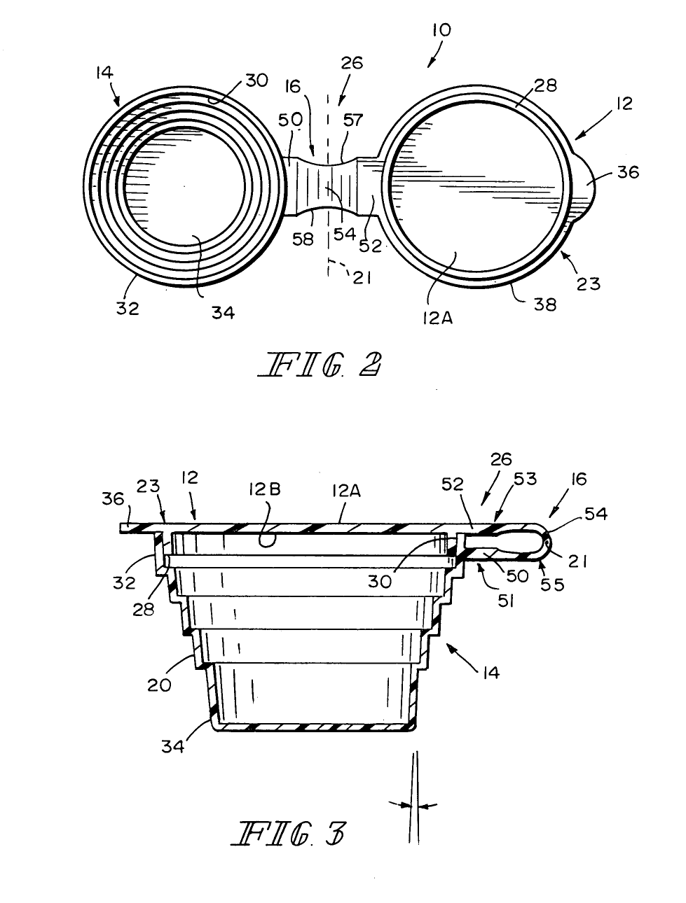

To close cup 14 when medicine (not shown) is in medicine storage region 19 formed in cup 14, lid 12 will normally be rotated about a pivot axis 21 established by living hinge 16 and closed on cup 14 as suggested in FIGS. 2 and 3. However, when cup 14 is empty, either or both portions 12, 14 may be rotated about living hinge 16 to close lid 12 on cup 14.

As shown in FIG. 1, conical tapering 18 decreases from an upper part 32 of the cup 14 to abottom part 34 of the cup 14. It is within the scope of this disclosure to form cup 14 to have minimal conical tapering and therefore appear to be substantially uniformly cylindrical (not shown) or even have a tapering in a reverse direction (not shown) to the tapering 18 shown in FIGS. 1 and 3.

As shown in FIG. 2, lid 12 includes a round disk 23 that is generally circular in shape and flange 28 that is coupled to underside 12A of round disk 21 and has an annular shape. The shape of lid 12 may also be, for example, rectangular or pyramidal. The shape of lid 12 will generally conform to a shape of upper part 32 of cup 14. Lid 12 may include a tab 36 extending beyond an outer edge 38 of round disk 23 to assist a user, for example, in closing lid 12 on and/or separating lid 12 from cup 14. In other words, tab 36 may assist in closing and opening container 10.

As shown in FIG. 1, cup 14 may include at least one marking 15, in the form of capacity and content level indicia, on outer surface 22. The indicia may be on or adjacent at least one of stepped increments 20. Cup 14 may also be adapted to be markable with other than content level indicia, such as, for example, user identification and/or dosage information (not shown). All or a portion of outer surface 12B of lid 12 may be adapted to be markable, using, for instance, a writing instrument to record, for example, patient or user identification and/or dosage information (not shown). Container 10 of the present disclosure may be disposable or reusable.

In the embodiment of FIG. 1, the monolithically incorporated living hinge 16, among other things, helps prevent loss of lid 12. Stepped increments 20, among other things, help a user identify or confirm capacity levels of container 10 by touching or feeling the outer side wall 24.

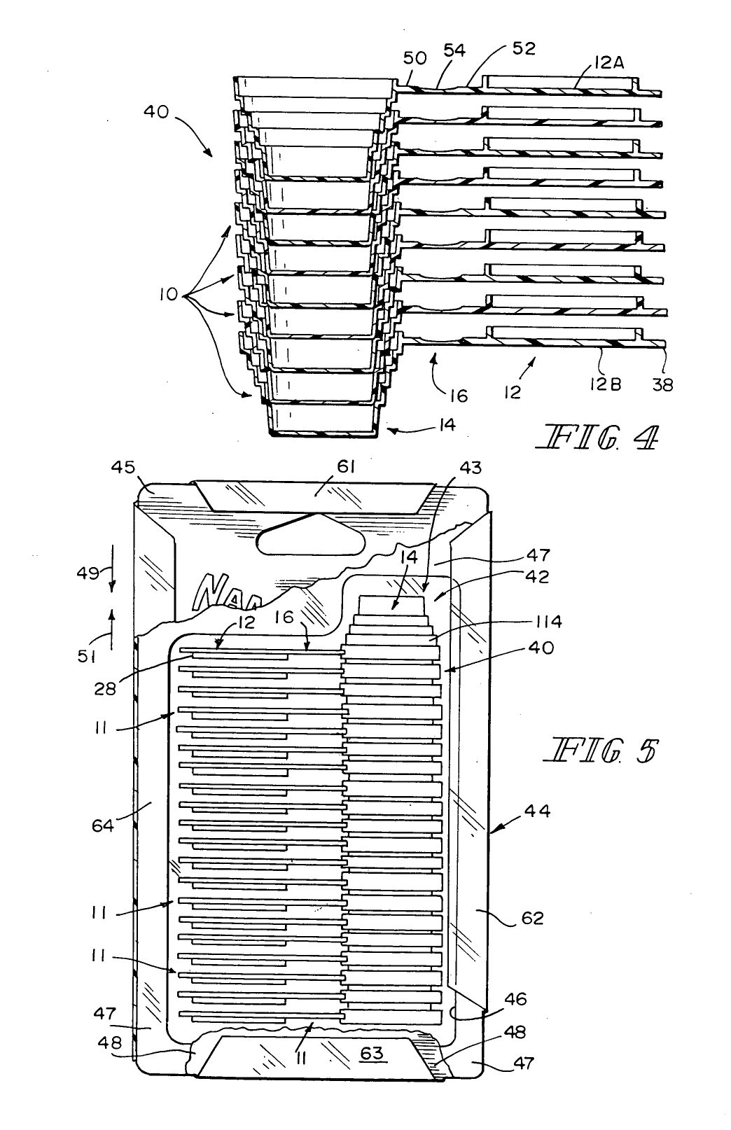

Conically-tapered configuration 18 of cup 14 may permit a nesting of a plurality of containers 10. Cups 14 in FIGS. 4 and 5 are shown as a nested stack 40, with containers 10 in an opened condition. Cups 14 are nested one inside the other and lids 12 are arranged to lie one adjacent to the other. The stack of nested cups 40 is upside down, that is upper part 32 of cups 14 are shown facing in a downward direction, toward the bottom of the page as one views FIG. 4. The stack of cups 40 can also be right-side up, with the bottom part 36 of the cups 14 facing in a downward direction (not shown). Alternatively, the containers 10 may be stackable (but not nestable) in a closed condition (not shown), with bottom part 36 of cup 14 of one container 10 placed on top of outer surface 12B of lid 12 of another container 10, or, vice versa. Further, the stack of nested cups 40 may be stacked such that lids 12 are in a fan-like configuration (not shown) or lids 12 are arranged at approximately a 90° angle to cups 14 (not shown).

A nested stack of cups 40, or non-nested stack (not shown) is insertable in a package 42, as shown in FIG. 5. Package 42 may be a blister pack (not shown) or sealed in an equivalent manner (not shown). The package may be re-sealable and reusable. Package 42 shown in FIG. 5 is not sealed.

As suggested in FIGS. 1-3, container 10 includes a medicine cup 14 formed to include a medicine storage chamber 19 having an open mouth 13 and a cantilevered member 11 coupled to medicine cup 14. Cantilevered member 11 includes a lid 12 configured to mate with medicine cup 14 to close open mouth 13 and a tether 16 arranged to interconnect medicine cup 14 and lid 12. Tether 16 is configured to provide a living hinge therebetween to tether lid 12 to medicine cup 14 during movement of lid 12 from an opened position (shown, for example, in FIGS. 1 and 2) extending laterally away from the medicine cup to a closed position (shown, for example, in FIG. 3) mating with medicine cup 14 to close the opened mouth 13.

As suggested in FIGS. 2 and 3, tether 16 is a strip of material having a first end 50 having a first thickness 51 appended to medicine cup 14, an opposite second end 52 having a second thickness 53 appended to round disk 23 of lid 12, and a middle portion 54 interconnecting first and second ends 50, 52. Middle portion 54 has a thickness 55 that is thinner than first and second thickness 51, 53. Middle portion 54 of tether 16 includes a first concave side edge wall 57 arranged to extend between first and second ends 50, 52 and lie in spaced-apart relation to the first side wall 57 as shown best in FIG. 2. In the illustrated embodiment, first thickness 51 is about equal to second thickness 53. Also, round disk 23 has a thickness 56 that is about equal to thickness 53 of second end 52 of tether 16.

As suggested in FIGS. 1 and 2, lid 12 includes a round disk 23 of material coupled to second end 52 of tether 16 and a flange 28 coupled to round disk 23. Flange 28 is sized to extend into open mouth 13 of medicine storage chamber 19 and mate with an upper edge 30 of medicine cup 14 upon movement of lid 12 to the closed position as shown in FIG. 3.

Medicine cup 14, tether 16, and lid 12 are made of a plastics material to form a monolithic container 10. Tether 16 is made of a pliable plastics material having a stiffness sufficient to support lid 12 in a horizontally extending cantilevered position upon movement of lid 12 to the opened position as shown, for example, in FIGS. 1 and 4.

A system is disclosed herein for storing and dispensing medicines. As suggested in FIG. 5, the system comprises a package 42 formed to include a cup storage region 43. A series of cups 14 is nested together to form a stack of cups and is located in the cup storage region 43. Each cup 14 is formed to include a medicine storage chamber 19 having an open mouth 13. All but one of cups 14 is arranged to extend into medicine storage chamber 19 of an adjacent cup 14 through open mouth 13 thereof as suggested in FIGS. 4 and 5.

Package 42 includes a body 44 formed to include cup storage region 43 and a cover 45 coupled to body 44 to close an access opening 46 into cup storage region 43. Cover 45 is mated to body 44 for repeated movement between a first position (shown in FIG. 5) closing access opening 46 and other positions (not shown) opening access opening 46. Means is provided for coupling cover 45 to body 44 for repeated movement between a first position closing access opening 46 into cup storage region 43 and other positions opening access opening 46.

Body 44 includes a tub 47 formed to include cup storage region 43 and flaps 61, 62, 63, 64 coupled to edges of tub 47 to provide means for retaining cover 45 on tub 47 in a position closing access opening 46 while the series of cups 14 is located in cup storage region 43 and for allowing sliding movement of cover 45 relative to tub 47 to uncover access opening 46 to permit removal of at least one of containers 10 from cup storage region 43 formed in tub 47 through access opening 56 without removal of all of containers 10 from cup storage region 43. Cantilevered members 11 are retained in spaced-apart relation to one another while containers 10 are located in cup storage region 43 owing to a stiffness of property of each cantilevered member 11.

To access containers 10 in package 42, a user might, for example, bend flap 63 outwardly relative to tub 47 to release a lower portion 48 of cover 45 from a normally trapped position between tub 47 and flap 63. Now, flap 63 lies between tub 47 and lower portion 48 of cover 45. Once released, cover 45 can be moved downwardly in direction 49 (see FIG. 5) along a “guide channel” established by opposing flaps 62, 64 to disengage flap 61 and “uncover” access opening 46. A consumer could then remove a first container 114 from cup storage region 43 and also remove other containers 14 from that region 43. Movement of cover 45 in opposite direction 51 functions to cover access opening 46 to retain any remaining containers 10 in cup storage region 43.

Although the present disclosure has been described and illustrated in detail, it is to be clearly understood that this is done by way of illustration and example only and is not to be taken by way of limitation. The spirit and scope of the present disclosure are to be limited only by the terms of the appended claims.

1. A monolithic container for storing and dispensing liquid medicines, the container comprising
a lid,
a cup connected to the lid by a monolithically incorporated living hinge,
the cup having a generally tapered configuration and having a plurality of spaced-apart stepped increments on an outer surface of a side wall indicating capacities and content levels of the cup, and
a securing mechanism monolithically incorporated with the container, and releasably securing the lid and cup when at least one of the lid and cup is rotated about the living hinge and closed on the other.

2. The container of claim 1, wherein the tapering is conical.

3. The container of claim 1, wherein the plurality of stepped increments are located at least partially around the outer surface of the side wall.

4. The container of claim 1, wherein the plurality of stepped increments are located completely around the outer surface of the side wall.

5. The container of claim 1, wherein the plurality of stepped increments are raised ribs.

6. The container of claim 2, wherein the plurality of stepped increments are raised ribs.

7. The container of claim 3, wherein the plurality of stepped increments are raised ribs.

8. The container of claim 1, wherein the conical tapering decreases from a top of the cup to a bottom of the cup.

9. The container of claim 1, wherein the securing mechanism includes at least one interlocking element on each of the lid and the cup.

10. The container of claim 9, wherein the interlocking elements include a recess on the lid and a flange on the cup.

11. The container of claim 9, wherein the interlocking elements include a recess on the cup and a flange on the lid.

12. The container of claim 1, wherein the lid includes a tab extending beyond an outer edge of the lid.

13. The container of claim 1, wherein the cup includes at least one indicia marked on the outer surface of the side wall, the indicia being located one of on and adjacent the stepped increments.

14. The container of claim 13, wherein the indicia indicate capacity and content levels of the cup.

15. The container of claim 1, wherein the lid includes an outer surface adapted to be markable.

16. The container of claim 1, wherein the container is made from moldable plastic.

17. The container of claim 1, wherein the container is injection molded.

18. The container of claim 1, wherein the lid is generally circular.

19. The container of claim 1, wherein the lid is shaped to match a shape of an upper part of the cup.

20. The container of claim 1, wherein the container is disposable.

21. The container of claim 1 in combination with additional containers, nested together in at least one stack.

22. The container of claim 1 in combination with additional containers, nested together in at least one stack and the at least one stack being sealed in a package.

23. The container of claim 22, wherein the package is resealable.

24. A container for storing and dispensing liquid medicines, the container comprising
a medicine cup formed to include a medicine storage chamber having an open mouth, and
a cantilevered member coupled to the medicine cup, the cantilevered member including a lid configured to mate with the medicine cup to close the open mouth and a tether arranged to interconnect the medicine cup and the lid and configured to provide a living hinge therebetween to tether the lid to the medicine cup during movement of the lid from an opened position extending laterally away from the medicine cup to a closed position mating with the medicine cup to close the opened mouth.

25. The container of claim 24, wherein the tether is a strip of material having a first end having a first thickness appended to the medicine cup, an opposite second end having a second thickness appended to the lid, and a middle portion interconnecting the first and second ends, and the middle portion has a thickness that is thinner than the first and second thicknesses.

26. The container of claim 25, wherein the first thickness is about equal to the second thickness.

27. The container of claim 25, wherein the lid includes a round disk of material coupled to the second end of the tether and a flange coupled to the round disk and sized to extend into the open mouth of the medicine storage chamber and mate with an upper edge of the medicine cup upon movement of the lid to the closed position.

28. The container of claim 27, wherein the round disk has a thickness that is about equal to the thickness of the second end of the tether.

29. The container of claim 25, wherein the middle portion of the tether includes a first concave side edge wall arranged to extend between the first and second ends and a second concave side edge wall arranged to extend between the first and second ends and lie in spaced-apart relation to the first side wall.

30. The container of claim 24, wherein the medicine cup, tether, and lid are made of a plastics material to form a monolithic container.

31. The container of claim 24, wherein the tether is made of a pliable plastics material having a stiffness sufficient to support the lid in a horizontally extending cantilevered position upon movement of the lid to the opened position.

32. A system for storing and dispensing medicines, the system comprising
a package formed to include a cup storage region and
a series of cups nested together to form a stack and located in the cup storage region, each cup being formed to include a medicine storage chamber having an open mouth, and wherein all but one of the cups is arranged to extend into the medicine storage chamber of an adjacent cup through the open mouth thereof.

33. The system of claim 32, wherein the package includes a body formed to include the cup storage region and a cover coupled to the body to close an access opening into the cup storage region.

34. The system of claim 33, wherein the cover is mated to the body for repeated movement between a first position closing the access opening and other positions opening the access opening.

35. The system of claim 34, wherein the body includes a tub formed to include the cup storage region and flaps coupled to edges of the tub to provide means for retaining the cover on the tub in a position closing the access opening while the series of cups is located in the cup storage region and for allowing sliding movement of the cover relative to the tub to uncover the access opening to permit removal of at least one of the containers from the cup storage region formed in the tub through the access opening without removal of all of the containers from the cup storage region.

36. The system of claim 32, wherein the package includes a body formed to include the cup storage region, a cover, and means for coupling the cover to the body for repeated movement between a first position closing an access opening into the cup storage region and other positions opening the access opening.

37. The system of claim 32, wherein a cantilevered member is coupled to each of the cups, each cantilevered member includes a lid configured to mate with one of the cups to close the open mouth thereof and a tether arranged to interconnect the companion cup and lid to provide a living hinge therebetween and the cantilevered members are located in the cup storage region in side-by-side relation to the nested cups to form a stack of lids.

38. The system of claim 37, wherein the cantilevered members are retained in spaced-apart relation to one another while the containers are located in the cup storage region owing to a stiffness property of each cantilevered member.
